(19) United States
(12) Patent Application Publication
(10) Pub. No.: US 2010/0071690 A1
(43) Pub. Date: Mar. 25, 2010

(54) Face warming breath deflecting apparatus

(76) Inventor: Ronald D. Matich, Baxter, MN (US)

Correspondence Address: ROBERT J JACOBSON PA, 650 BRIMHALL STREET SOUTH, ST PAUL, MN, 551161511, US

(21) Appl. No.: 12/321,632

(22) Filed: Jan. 23, 2009

Related U.S. Application Data
(63) Continuation-in-part of application No. 12/284,651, filed on Sep. 23, 2008.

(51) Int. Cl. A61M 16/06

(52) U.S. Cl. 128/203.12

(57) ABSTRACT

A thermally active breath deflecting apparatus having a flexible base of sufficient width to extend at least to and between the corner portions of the eyes and of sufficient length to extend at least from immediately below the eyes to at least beyond the tip of the nose such that breath from the mouth and nose is prevented from traveling upwardly over the eyes. The apparatus includes a thermal pack that can warm or cool the face. The thermal pack is preferably a heat pack. In an alternate embodiment, the flexible base can include medication such as a decongestant.

This application is a continuation-in-part of U.S. patent application Ser. No. 12/284,651 filed Sep. 23, 2008 and claims the benefit of such application under 35 U.S.C. 120. U.S. patent application Ser. No. 12/284,651 is incorporated by reference in its entirety into this application.

### FIELD OF THE INVENTION

The present invention generally relates to breath deflectors, and specifically relates to breath deflectors having thermal packs to warm or cool a portion of the face.

### BACKGROUND OF THE INVENTION

A breath deflector is an apparatus that deflects breath away from the eyes. A breath deflector is an apparatus placed between 1) the nose and mouth and 2) the eyes. For example, a motorcycle rider may wear glasses inside of a helmet. As the rider breathes from the mouth and the nose, his or her breath may rise to form a mist on his or her glasses, making it difficult for the motorcycle rider to see. When a barrier, such as a breath deflector, is placed between 1) the eyes and 2) the mouth and nose, then moist breath rises around and away from the eyes and eyewear and is dispersed to the atmosphere as a whole. As to a breath deflector, the Matich U.S. Pat. No. 5,797,146 issued Aug. 25, 1998 and entitled Breath Deflector With Seal is hereby incorporated in reference in its entirety.

A thermal pack can be hot or cold. A thermal pack is an apparatus that can be placed on a portion of the body to warm up or cool down the portion of the body. A thermal pack can be cooled over time in a refrigerator or freezer. A thermal pack can be of the type that is filled with hot, warm, or cold water. A thermal pack can generate heat as a result of an exothermic chemical reaction within the thermal pack. Or, as a result of an endothermic chemical reaction within the thermal pack, the thermal pack can become cool or cold to the touch.

### SUMMARY OF THE INVENTION

A feature of the present invention is the provision in a breath deflector, of a thermal pack for warming or cooling the face.

Another feature of the present invention is the provision in a breath deflector having a thermal pack, of a flexible base, and of a sticky elongate section on a rear side of the flexible base, where the sticky elongate section is releasably stickable to the skin, and where a width of the sticky elongate section is sufficient to extend at least to and between the outer corner portions of the eyes of an adult such that breath from the mouth and nose is prevented from traveling upwardly over the eyes.

Another feature of the present invention is the provision in a breath deflector having a thermal pack, of a flexible base, and of the flexible base having a length sufficient to extend from the eyes to at least the tip of the nose along the bridge of the nose of an adult.

Another feature of the present invention is the provision in a breath deflector having a thermal pack, of the thermal pack being engagable to and disengagable from the breath deflector.

Another feature of the present invention is the provision in a breath deflector having a thermal pack, of the thermal pack being oriented along the width and length of the flexible base and is disposed generally medially between the sides of the flexible base such that said thermal pack extends along the bridge of the nose.

Another feature of the present invention is the provision in a breath deflector having a thermal pack, of the thermal pack being sandwiched between a sticky portion of the flexible base of the breath deflector and a liner of the flexible base, and of the liner being perforated to permit air to maintain activation of the thermal pack.

Another feature of the present invention is the provision in a breath deflector having a thermal pack, of first and second sticky elongate sections on the rear side of the flexible base, of first and second liners, of each of said first and second liners covering one of said sticky elongate sections, and of each of the first and second liners being separately peelable.

Another feature of the present invention is the provision in a breath deflector, of the breath deflector having a flexible base, where an upper edge of the flexible base includes a middle portion, where the middle portion curves outwardly, and where the upper edge includes a pair of inwardly curving portions, with one of the inwardly curving portions being between the middle portion and the right edge, and with the other of the inwardly curving portions being between the middle portion and the left edge, such that the middle portion runs over the bridge of the nose, and such that the inwardly curving portions confront and track the outwardly curving portions of the eyes.

Another feature of the present invention is the provision in a breath deflector, of the breath deflector having a flexible base, where the lower edge of the flexible base includes a middle portion and a pair of curved extensions, where the middle portion is disposed inwardly relative to the curved extensions, where the lower edge includes a pair of tapering portions, where one of the tapering portions tapers from one of the curved extensions to a right edge of the flexible base, and where the other of the tapering portions tapers from the other of the curved extensions to the left edge of the flexible base.

Another feature of the present invention is the provision in a breath deflector, of an upper edge of the flexible base including a middle portion curving outwardly, of a lower edge of the flexible base including a middle portion disposed inwardly relative to a pair of curved extensions, and of the middle portions being aligned medially with each other so as to define for a user a center position on the flexible base for placement over the nose.

An advantage of the present invention is a warming of the face and a deflecting of the breath at the same time.

Another advantage of the present invention is that the breath deflector provides a ready made base for engaging a thermal pack.

Another advantage of the present invention is that the breath deflector engages the thermal pack without hindering the chemical reaction in the thermal pack.

Another advantage of the present invention is that the breath deflector, with or without the thermal pack, is inexpensive to manufacture.

Another advantage of the present invention is that the breath deflector, with or without the thermal pack, is simple to apply to the face and simple to take off.

Another advantage is that the breath deflector portion of the apparatus works to capture and contain heat generated by the thermal pack.

Another advantage of the breath deflector having the heat pack is that such apparatus is disposable.

Another advantage of the breath deflector having the heat pack is that such apparatus is safely disposable.

### BRIEF DESCRIPTION OF THE DRAWINGS

FIG. 1A is a perspective view of the present face warming breath deflecting apparatus, showing three rear liners at least partially peeled from the underlying flexible base and the thermal pack ready to be engaged between the flexible base and the liner that is perforated.

FIG. 1B is a perspective view of the face warming breath deflecting apparatus of FIG. 1A showing the apparatus in an assembled configuration where the thermal pack is sandwiched between the flexible base and the perforated liner.

FIG. 2A is a perspective environmental view of the face warming breath deflecting apparatus of FIGS. 1A and 1B, where the apparatus extends at least to and between the outside corners of the eyes and at least to the tip of the nose and preferably about to the lower lip of the mouth.

FIG. 2B is a perspective view of the airtight container for the thermal pack of the face warming breath deflecting apparatus of FIGS. 1A and 1B, as the thermal pack is preferably air activated and undergoes an exothermic chemical reaction when,exposed to the atmosphere.

FIG. 3A is a top plan view of the front side of the breath deflector portion of the face warming and breath deflecting apparatus of FIGS. 1A and 1B.

FIG. 3B is a top plan view of the rear side of the breath deflector portion of the face warming and breath deflecting apparatus of FIGS. 1A and 1B.

FIG. 4A is a perspective view of an alternate embodiment of the face warming breath deflecting apparatus, showing three rear liners at least partially peeled from the underlying flexible base, and further showing a vapor generating jelly and vapor generating granules engaged to the underlying flexible base.

FIG. 4B is a perspective view of an airtight container for the face warming breath deflecting apparatus of FIGS. 4A to minimize exposure to air of the vapor generating jelly and vapor generating granules.

FIGS. 5A, 5B, 5C and 5D each shows a chimney or tube portion of the flexible base or layer of apparatus 10 prior to use.

### DETAILED DESCRIPTION

Embodiment having a Thermal Pack

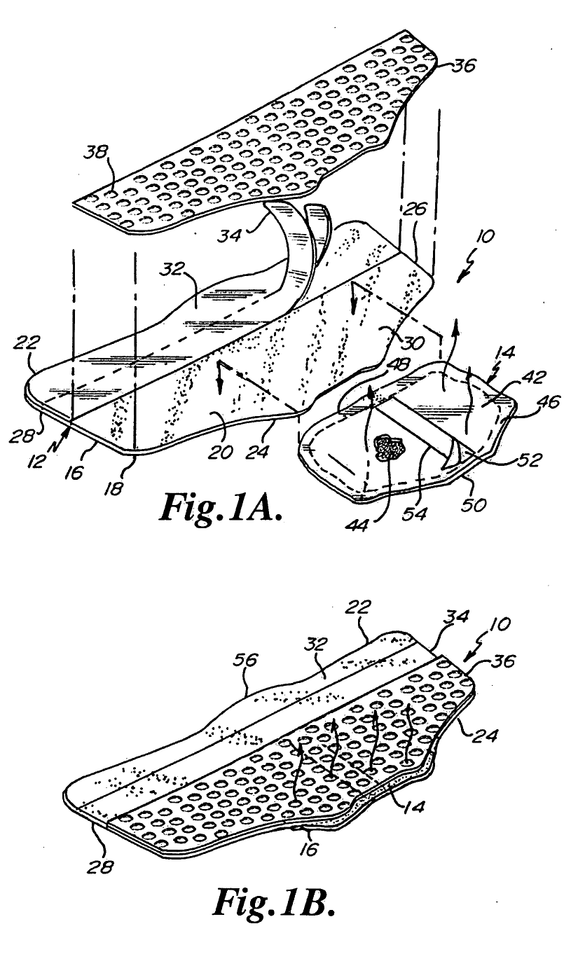

As shown in FIG. 1A, the present face warming and breath deflecting apparatus is indicated in general by reference number 10. Apparatus 10 includes a breath deflector 12 and a pack 14, preferably a thermal or thermally active pack 14.

Breath deflector 12 includes a flexible base 16. Base 16 includes a front side or face 18 and a rear side or face 20 defining a thickness therebetween, an upper edge 22 and a lower edge 24 defining a length therebetween, and a right edge 26 and a left edge 28 defining a width therebetween. The breath deflector 12 is generally formed in the shape of a set of wings or a flying wing.

The rear side 20 of the base 16 is generally entirely covered with a skin friendly adhesive 30 from upper edge 22 to lower edge 24 and from right edge 26 to left edge 28. The adhesive 30 is in turn covered by a first or upper peelable liner 32 that runs laterally, a second or middle peelable liner 34 that runs laterally and a third or lower peelable and perforated liner 36 that runs laterally.

The first peelable liner 32 is elongate and confronts the upper edge 22 of the base 16. The first peelable liner 32 extends from the right edge 26 to the left edge 28 of the base 16 and includes an upper edge that is flush with and follows the line of the upper edge 22 of the base 16. The lower edge of the first peelable liner 32 is straight. The end edges of liner 32 are rounded.

The second peelable liner 34 is elongate and confronts each of the first liner 32 and third liner 34. Liner 34 is disposed between first liner 32 and third liner 34. Liner 34 extends from the right edge 26 to the left edge 28 of the base 16. Liner 34 includes a straight upper edge and a straight lower edge. The end edges of liner 34 are straight and taper outwardly from the lower edge to the upper edge of liner 34.

The third peelable liner 36 is elongate and confronts the lower edge 24 of the base 16. The third peelable liner 36 extends from the right edge 26 to the left edge 28 of the base 16 and includes a lower edge that is flush with and follows the line of the lower edge 24 of the base 16. The upper edge of the third liner 36 is straight. The end edges of the liner 36 include a slight inwardly curving portion or recession or concavity.

The third peelable liner 36 is perforated from its upper to lower edges and from its right to left edges, with the perforations or holes 38 dispersed generally evenly throughout the liner 36. The purpose of the holes 38 is to let air to the thermal pack 14 when the thermal pack 14 is sandwiched between the base 16 and the third peelable liner 36. Thermal pack 14 may include chemicals that undergo a chemical reaction when exposed to air. If the air supply is cut off, the chemical reaction may slow down or stop.

Flexible base 16 is preferably a foam and is insulative. Liners 32, 34 and 36 are preferably formed of paper with a slippery underside that can readily stick to and be readily disengaged from adhesive 30.

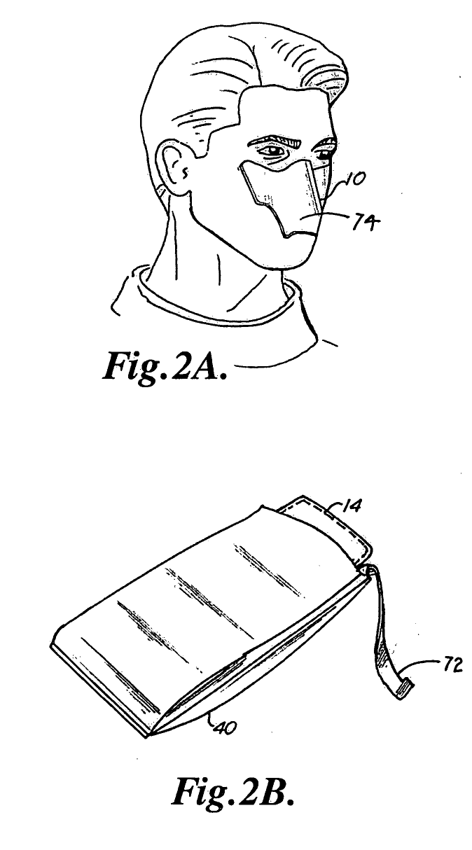

Prior to use, thermal pack 14 is contained in a sealed metal foil package 40, shown in FIG. 2B. If desired, apparatus 10 including the breath deflector 12 and thermal pack 14 may be contained in the metal foil package 40, shown in FIG. 2B, such that exposure to air of the adhesive 30 underlying the perforated liner 36 is minimized and such that any chemical reaction in the thermal pack 14 is minimized or uninitiated until the package 40 is opened. If desired, if shipped and stored in the metal foil package 40, thermal pack 14 may be separate from or already sandwiched in place between base 16 and perforated liner 36.

The thermal pack 14 includes an envelope 42 formed of a soft paper or plastic. The envelope 42 is pervious to air but impervious to chemicals 44 contained therein. The envelope 42 has a flat permanent or welded seal 46 formed about its periphery to keep the chemicals 44 contained therein unless the integrity of the envelope 42 is destroyed. The thermal pack 14 includes an upper or laterally running edge 48 and a lower or laterally running edge 50 and includes a strip of skin friendly adhesive 52 running from the upper edge 48 to the lower edge 50 on the rear side or face of the thermal pack 14. Adhesive 52 is covered by a peelable liner 54. The front side or face of the thermal pack 14 includes no strip of adhesive.

The thermal pack 14 may be an instant heat pack that is activated automatically by being exposed to air. The thermal pack 14 may contain a mixture of iron or iron powder, water, cellulose, activated carbon and salt. The mixture may also contain vermiculite. Once the outer package 40 is opened, which may be formed of a metal foil or other material that is relatively impermeable to air, an oxidation (rusting) process takes place which causes a safe, natural reaction to release heat (an exothermic reaction). The thermal pack 14 may stay warm for about six hours to 12 hours. Some packs are activated (exposed to oxygen) by the removal of a protective strip (instead of being contained in a package).

The thermal pack 14 may alternately be a pack that is cooled over time in a refrigerator or freezer. Or the thermal pack 14 can be of the type that is filled with hot, warm, or cold water or warmed in a microwave prior to use. The thermal pack 14 can generate heat as a result of an exothermic chemical reaction. Or, as a result of an endothermic chemical reaction, the thermal pack 14 can become cool or cold to the touch.

As shown in FIGS. 1A and 1B, thermal pack 14 is engaged to the breath deflector 12 by the steps of a) peeling away liner 54 from the strip of adhesive 52 on the thermal pack 14, b) peeling the third peelable liner 36 away from the flexible base 16 to expose a sticky portion of the adhesive 30, c) placing the front side or face of the thermal pack 14 on the adhesive 30 that was exposed by peeling away liner 36 such that the upper edge 48 of the thermal pack 14 confronts the lower edge of the second peelable liner 34 and such that the lower edge 50 of the thermal pack 14 is generally within or above the lower edge 24 of the base 16, d) placing the third peelable liner 36 back onto the flexible base 16 such that right and left portions of the underface of the liner 36 reengage the adhesive 30 and such that the remaining portion of the underface of the liner 36 engages the back side or back face of the thermal pack 14 and the strip of adhesive 52, then e) pressing upon the liner 36 such that 1) adhesive engagement between the adhesive 30 and liner 36 is maximized, 2) adhesive engagement between the adhesive 30 and the front face of the thermal pack 14 is maximized and 3) adhesive engagement between the adhesive 52 and the liner 36 is maximized. Adhesives 30 and 52 are releasable adhesives relative to paper, plastic and the face, such that the thermal pack 14 may be released from adhesive 30 and repositioned and such that the third liner 36 may be repositioned and reengaged again.

When one or more of first and second liners 32 and 34 are removed from the flexible base 16, a sticky elongate section of adhesive 30 is exposed on the rear side 20 of the flexible base 16. This sticky elongate section of adhesive 30 is releasably stickable to the skin. This sticky elongate section of adhesive 30 includes a width running from the right edge 26 to the left edge 28 of the flexible base 16, and such width is sufficient to extend at least to and between the outer corner portions of the eyes of an adult such that breath from the mouth and nose is prevented from traveling upwardly over the eyes, as shown in FIG. 2A.

It should be noted that one, two or three of liners 32, 34, and 36 may be removed from the base 16, whereupon the thermal pack 14 may be stuck to generally any portion of the adhesive 30 on the sticky face of the base 16 so as to leave a lateral strip of exposed adhesive along the upper edge 22 of the base 16 so as to form a lateral continuous seal to the skin directly underneath the eyes, whereupon the removed liners can be reapplied to the base 16 and over the thermal pack 14.

The base 16 includes a length running from the upper edge 22 to the lower edge 24. This length is sufficient to extend from the eyes, or just below the eyes such as at the eye sacs, to at least the tip of the nose along the bridge of the nose of an adult. A length axis of the thermal pack 14 is oriented along and aligned with a length axis of the flexible base 16 and is disposed generally medially between the sides of the flexible base 16 such that the thermal pack 14 extends along the bridge of the nose. The length of the base 16, or the length of the apparatus 10, runs preferably from a point on the bridge of the nose just under the level of the eyes to a point beyond the tip of the nose, or to a point at about the lower lip, or to a point between the mouth and the chin.

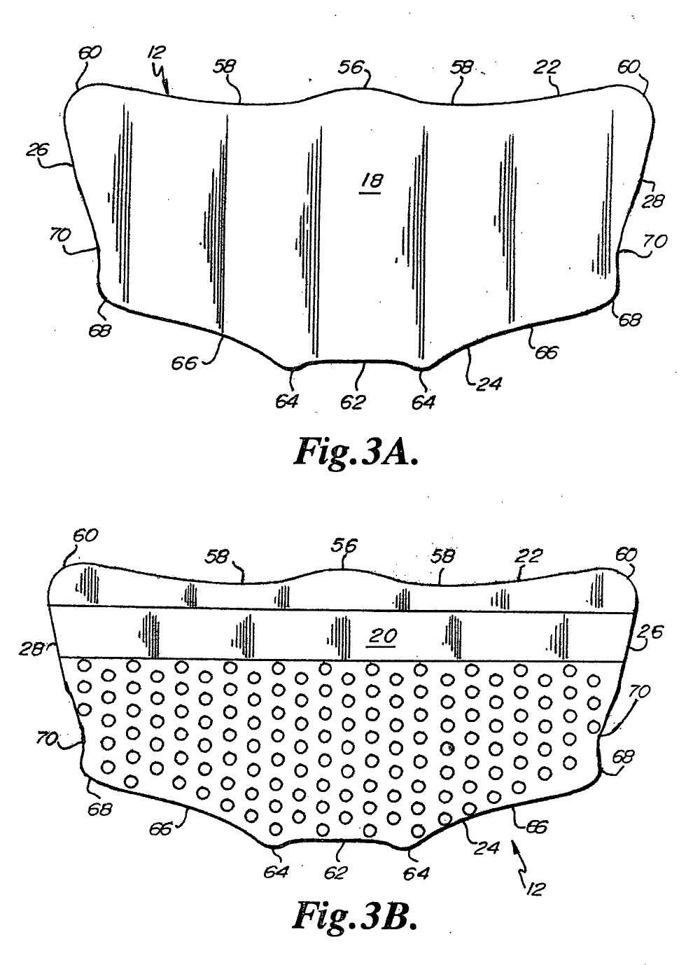

As shown in FIGS. 3A and 3B, the upper edge 22 of the flexible base 16 includes a convex middle portion 56 that is curved or rounded outwardly. The upper edge 22 further includes a pair of inwardly curving or rounded concave portions 58. One of the inwardly curving portions is between the middle portion 56 and the right edge 26. The other of the inwardly curving portions is between the middle portion 56 and the left edge 28 such that the middle portion 56 runs over the bridge of the nose, and such that the inwardly curving portions confront the eyes, particularly the outwardly curvature of the eyes. Upper edge 22 has rounded corners or junctions 60 with respective right and left edges 26, 28.

The lower edge 24 of the flexible base 16 includes a middle portion 62. The middle portion 62 is disposed inwardly relative to a pair of curved convex extensions 64 and extends in a straight linear fashion to and between the curved extensions 64. Middle portion 62 is opposite the crest of the middle portion 56 of the upper edge 22. The lower edge 24 further includes a pair of tapering and inwardly curving or slightly concave portions 66. Each of the tapering and inwardly curving portions 66 is disposed between one of the curved extensions 64 and a rounded corner or junction 68. Rounded corner 68 is a junction with one of the right and left edges 26, 28.

Each of the right edge 26 and left edge 28 of the flexible base 16 includes an inwardly extending rounded or curved or concave portion 70. From the innermost portion of concave portion 70, each of the right and left edges 26, 28 tapers outwardly to junctions 60 to provide a flying wing shape to the flexible base 16 or to the apparatus 10 as a whole, such that a distance between upper junctions 60 is greater than a distance between lower junctions 68.

The middle portions 56 and 62 are aligned medially with each other so as to define for a user a center position on the flexible base 16 for placement over the nose. Curved extensions 64 also provide a centering function for the user. Inwardly curved portions 58 track the outwardly curving portions of the eyes.

It should be noted that a piece of cloth or fabric, such as cheesecloth, may be included in the package 40 with the thermal pack 14 and the breath deflector 12. This piece may be generally the length and width of the thermal pack 14. This piece can be placed between the thermal pack 14 and the liner 36. The purpose of the piece is to absorb some of the heat of the thermal pack 14 such that less heat is conveyed to the face of the user. This piece may be defined as an insulative piece. This piece can be held in place by adhesive 52 and can be further held in place by the sandwiching action of the liner 36.

In operation, a strip 72 is torn off package 40 to open package 40 for removal of the thermal pack 14. At such a point in time, thermal pack 14 is exposed to air, is thus activated, and begins to generate heat. Liner 36 is removed. The thermal pack 14 is engaged to the sticky portion of the adhesive 30 that was exposed by the removal of the liner 36. Liner 50 is removed to expose adhesive 52. Then the liner 36 is reengaged to the right and left sides of the sticky portion of the adhesive 30 that are still exposed. At the same time, the liner 36 is engaged to the adhesive 52 on the thermal pack 14. Then one or more of the first and second liners 32, 34 are removed, exposing an elongate sticky section of adhesive 30 that is applied to the skin of the face over the bridge of the nose and generally directly under the eyes and to just beyond the corner of the eyes. In such a position on the face, where the breath deflector 12 extends at least to and between the outside corners of the eyes, and where the breath deflector 12 extends at least to the tip of the nose, the nose and cheeks are warmed by the thermal pack 14 and breath is diverted away from the eyes and thus diverted away from glasses being worn in front of the eyes. Breath deflector 12 also acts to contain heat from the thermal pack and redirect such heat from the nose to and along the cheeks. To remove the breath deflector 12 having the thermal pack 14, the flexible base 16 is grabbed and pulled, thereby peeling the elongate section of adhesive 30 away from face.

It should be noted that the breath deflector 12 includes the first or upper liner 32 that covers a sticky elongate adhesive section of adhesive 30 where such adhesive section has a width sufficient to extend to and between the outer corner portions of the eyes. It should be noted that the breath deflector 12 further includes the second or lower or inner or medial liner 34 that covers a sticky elongate adhesive section of adhesive 30 where such adhesive section has a width sufficient to extend to and between the outer corner portions of the eyes. The preferred way to use the separately peelable first and second liners 32, 34 is to first remove the first upper liner 32 to expose the respective section of adhesive 30, which adhesive section is then applied to the face to and between the outer corner portions of the eyes directly under the eyes. Then, such as during a snowmobile race, or during a motorcycle race, or a snowmobile trip, or other event or activity, when the user's face becomes dirty or sweaty, when the adhesive 30 begins to lose its adhesion to the face, when the breath deflector 12 has been on the face for a long time, or when the breath deflector 12 has been taken off such as over a lunch period, the second lower liner 34 may be peeled off to expose a fresh section of adhesive 30. This fresh section of adhesive 30 then provides added adhesive strength to hold the apparatus 10 in place.

It should be noted that perforated liner 36 may be referred to as a rate and temperature controller of the thermal pack 14. In other words, perforations 38 may vary in diameter, in position, and in number to regulate the amount of air flowing to the thermal pack 14. For example, the rate of an exothermic reaction in the thermal pack 14, or the temperature of the thermal pack 14, may increase with a greater number of perforations 38 or with perforations of a greater diameter. Or the thermal pack 14 may have a longer life span, providing heat over a greater period of time, where the perforations 38 are of a smaller diameter or are fewer in number. Perforations 38 may vary in diameter and position within one apparatus 10. Perforations 38 may vary in diameter, position, and in number from one apparatus 10 to another apparatus 10.

Embodiments Having Medication

In an alternate embodiment of the invention, chemicals or composition 44 in FIG. 1A can indicate a medication. A medication 44 can be a substance or composition that can be used for curing, mitigating, treating, or preventing a condition or disease. A medication can be a drug.

As shown in FIG. 1A, medication 44 is contained in pack 14, with or without chemicals that can generate heat or cold. Preferably medication 44 is solely contained in pack 14 without any chemical that can generated heat or cold.

As shown in FIG. 1B, pack 14 can be sandwiched between flexible base 16 and perforated liner 36, with the medication 44 in the pack 14.

As shown in FIG. 2A, apparatus 10 can be applied to the nose and shaped in the form of a chimney or half-chimney or partial chimney, where a portion of the chimney is formed by the face, such as the upper lip, and the remainder of the chimney is formed by the apparatus 10. Apparatus 10 can run from a point on the bridge of the nose just under the level of the eyes to a point beyond the tip of the nose, or to a point at about the lower lip, or to a point between the mouth and the chin.

As shown in FIG. 2B, the medication 44 can be contained in a sealed package so as to minimize, prior to use, exposure of the medication 44 to air.

As shown in FIGS. 3A and 3B, apparatus 10 can have medial portions 56 and 62 that are aligned with each other so as to define for a user a center position for placement over the nose.

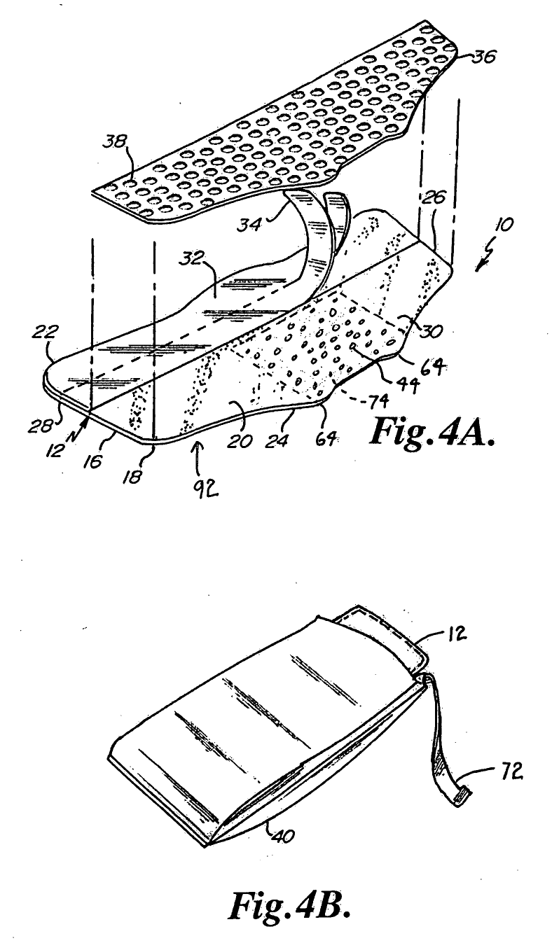

As shown in FIG. 4A, medication 44 can be engaged on or within the flexible layer 16 in an area 74 having longitudinal boundaries running vertically from curved extensions 64, an upper lateral boundary confronting the lower edge of liner 34, and a lower lateral boundary running along being colinear with lower edge 24 and further running to and being colinear with middle portion 62. Area 74 is referred to as a chimney forming portion. If desired, area 74 can be wider (have longitudinal boundaries running closer to and even confronting and running along sides edges 26 and 28. If desired, area 74 can be longer (have lateral boundaries closer to and even confronting and running along upper edge 22.

As shown in FIG. 4B, breath deflector portion 12 can be contained within a sealed metal foil package 40 that can be opened by a strip 72. Package 40 minimizes the medication 44 being exposed to air.

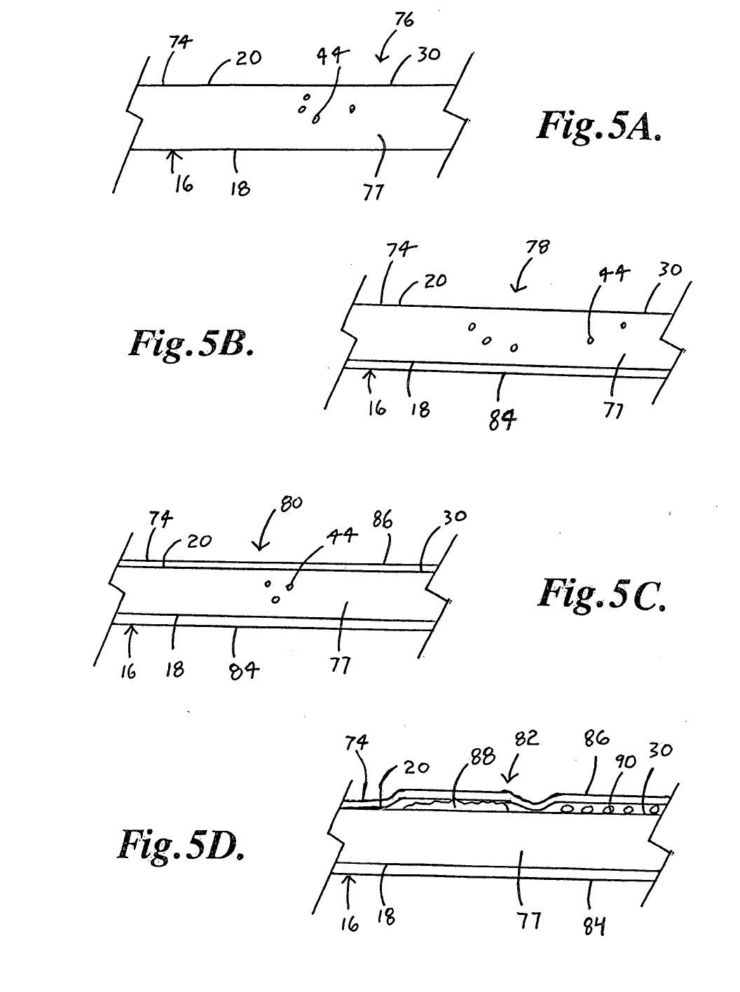

FIGS. 5A, 5B, 5C, and 5D show alternate embodiments 76, 78, 80 and 82 of the chimney area 74.

FIG. 5A shows an embodiment 76 where flexible layer 16 includes a material 77 having medication 44 therein. Material 77, such as open cell foam or other material having open cells, can be porous from the rear face 20 to the front face 18. Medication 44 in vapor or gas form can pass through either or both of the front and rear faces 18, 20 of material 77. Chimney portion 74 can have a porous adhesive on rear face 20, or medication 44 in layer 16 can be mixed with adhesive, or chimney portion 74 can have no adhesive and permit adhesive 30 that is outside of area 74 operate to fix the apparatus 10 to the face. Front face 18 points away from the face of the user. Rear face 20 points toward the face of the user.

FIG. 5B shows an embodiment 78 that is identical to embodiment 76, except that embodiment 78 includes a sealing layer 84 that minimizes transmission of vapor or gas from the medication 44 so as to direct the vapor and gas toward and out of the rear face 20. Sealing layer 84 is relatively permanent and is unpeelable.

FIG. 5C shows an embodiment 80 that is identical to the embodiment 78, except that embodiment 80 includes a liner or removable sealing layer 86 that minimizes the transmission of vapor or gas from the medication 44. Removable sealing layer or liner 86 can be peeled off apparatus 10 to expose the porous adhesive 30 and porous rear face 20. Whereas embodiments 76, 78 are contained in package 40 prior to use, embodiment 80 is stand alone because medication 44 is sealed between layers 84 and 86.

FIG. 5D shows an embodiment 82 that is identical to embodiment 80, except that the medication 44 is not contained within the material 77, but is engaged on the rear face of material 77, such as on adhesive 30 that may or may not be porous. In FIG. 5D, the spacing between material 77 and peelable layer 86 is exaggerated to show therebetween medication 44 in a gel form as shown by reference number 88 and medication 44 in a granular form as shown by reference number 90.

It should be noted that FIG. 4A shows an embodiment 92 identical to embodiment 86 of FIG. 5C, except that embodiment 92 includes a perforated liner 36 in place of the sealing layer 86. Prior to use, this embodiment 92 may be contained in package 40, and this medication may be somewhat exothermic when exposed to the air, such that perforated liner 36 spaces the exothermic medication slightly from the nose or face of the user.

It should be noted that the FIG. 1A, with or without the thermal pack 14, may be utilized with an off-the-shelf decongestant, where such an off-the-shelf decongestant if in the form of a gel can be applied to the rear face 20 of the flexible base 16, whereupon the perforated liner 36 can then be engaged over the gel decongestant, whereupon the apparatus 10 can be fixed to the face via removing one or more liners 32 and 34.

Desirably chimney portion 74 has medication in a single form, whether such as single form is a gel form, a granular form, liquid form, or a solid form. Embodiments 5A, 5B, and 5C can contain medication in a liquid form, where such liquid is contained within the cells of material 77.

Medication 44 can include one or more medications for the nose such as sympathomimetic, antihistamine, anticholinergic, NSAIDS, steroid, antiseptic, local anesthetic, antifungal, cerumenoloyti, and decongestant medications, where such medications can be prepared in a form that generates a vapor or gas when exposed to air.

Medication 44 can also include one or more medications for the respiratory system such as bronchodilator, NSAIDS, anti-allergic, antitussive, mucolytic, decongestant, corticosteriod, beta-receptor antagonist, anticholinergic, expectorant, and steroid medications, where such medications can be prepared in a form that generates a vapor or gas when exposed to air.

Medication 44 can be a vapor generating composition such as a decongestant to decongest sinus openings and passages, to promote sinus drainage, to open nasal passages, to relieve nasal congestion, and to provide symptomatic relief of nasal and sinus congestion.

The vapor generated by such medication 44 is intended to be administered through the nose (by an intranasal route).

Medication 44 can be medication that is applied topically. Medication 44 can be medication that may be somewhat abrasive to the skin because in such a case medication 44 can be covered by the perforated liner 36.

Medication 44 can be in a gel form, granular form, solid form, or liquid form and may be contained in a pharmaceutically acceptable carrier.

A decongestant may reduce swelling of mucous membranes. A decongestant can cause a response from an adrenoreceptor. A decongestant can also include an antihistamine to lessen the effect of an adrenoreceptor.

The decongestant can include one or more of the set of active components of menthol, camphor, eucalyptus oil, spirits of turpentine, aromatic oils, cedar leaf oil, myristica oil, mineral oil, nutmeg oil, and thymol, in a petrolatum base. The active components may be present in the decongestant in a total concentration of no greater than about 1% by weight of the decongestant. The total concentration of the active components may be from about 0.3% to about 1% by weight of the decongestant preparation. One set of active components may include from about 15 to about 25 menthol, from about 30 to about 40 camphor, from about 5 to about 10 eucalyptus oil, and from about 30 to about 40 spirits of turpentine.

It should be noted that front side 18 faces away from the body or away from the surface of the skin to which the flexible base 16 is engaged. It should be noted that rear side 20 faces the body and skin, confronts the body and skin, and may make contact with the body and skin.

Medication 44 may be mixed with a pressure sensitive skin friendly adhesive and engaged in the material 77 that makes up the rear side 20.

In operation, chimney portion 74 curves about and confronts the nostrils and tip of the nose of the user. Chimney portion 74 makes contact with the skin of the nose about the nostrils of the nose and at the tip of the nose. Chimney portion 74 extends beyond the tip of the nose such as to midway between the nose and the upper lip, such as to the upper lip, such as to the lower lip, and such as to a position between the lower lip and the chin. Since the rear side 20 of the chimney portion 74 faces the body and skin, rear side 20 confronts the open nostrils of the nose from a position above the nostrils of the nose.

The chimney portion 74 shown in FIGS. 4A, 5A, 5B, 5C and 5D engages the nose and can extend beyond the nose and beyond the tip and nostrils of the nose and can be the only portion of the apparatus 10 that is required for directing medication to and up the nasal passages. However, for support, remaining portions of the breath deflector portion 12 can be utilized to support the chimney portion 74.

Chimney portion 74 itself, in combination with the upper lip, forms a chimney about the nose, whereupon vapor is released and contained, and then passes up the chimney portion 74, and enters the nasal passages. The remaining portions of breath deflector portion 12 can be utilized to provide support for such a chimney, and this remaining portion can be stuck to, for example, cheek portions of the face or to portions of the face adjacent to the nose, or to the bridge of the nose and portions adjacent to the bridge of the nose.

Chimney portion 74 is a tube portion and can generally form a tube with the upper lip or the portion of the face above the upper lip and below the nose.

It should be noted that the portion of the rear side or face 20 may or may not be covered by perforated liner 36. If not covered by perforated liner 36, the corresponding portion of the rear side or face 20 sticks to the nose about the nostrils and the tip of the nose via the adhesive 30 to form the chimney portion 74 and further sticks to the nose and face via the adhesive underlining strips 32, 34, one or more of which may be removed. If covered by perforated liner 36, the corresponding portion of the rear side or face 20 does not stick to the portion of the nose about the nostrils and the tip of the nose, but this corresponding portion of the rear side or face 20 still forms the chimney portion 74 about the nostrils and the tip of the nose because the flexible base 16 takes on such a shape as one or more strips 32, 34 are removed and the flexible base 16 is shaped or bent or applied about the bridge of the nose.

It should be noted that the breath deflecting portion 12 extends to and between the outer corner portions of the eyes and thereby protects the eyes against vapors released by the medication 44, including decongestant medication.

As to apparatus 10, the following U.S. patents are hereby incorporated by reference in their entireties: 1) the Cilento et al. U.S. Pat. No. 4,427,737 issued Jan. 24, 1984 and entitled Microporous Adhesive Tape, 2) the Parker U.S. Pat. No. 4,738,984 issued Apr. 19, 1988 and entitled Antirhinovirus Agents, 3) the Bates U.S. Pat. No. 4,826,683 issued May 2, 1989 and entitled Decongestant, 4) the Bates U.S. Pat. No. 4,927,631 issued May 22, 1990 and entitled Decongestant Preparation, 5) the Kielley U.S. Pat. No. 5,114,979 issued May 19, 1992 and entitled Fruity Flavored Nasal Decongestant Composition, 6) the Morris U.S. Pat. No. 5,288,492 issued Feb. 22, 1994 and entitled Decongestant Composition Containing Aloe Vera, 7) the Hughes et al. U.S. Pat. No. 5,322,689 issued Jun. 21, 1994 and entitled Topical Aromatic Releasing Compositions, 8) the Geria U.S. Pat. No. 5,478,656 issued Dec. 26, 1995 and entitled Treatment Of Sinus Headache, and 9) the Block et al. U.S. Pat. No. 6,090,403 issued Jul. 18, 2000 and entitled Inhalation Therapy Decongestant With Foraminous Carrier.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalents of the claims are intended to be embraced therein.

I claim:

1. An apparatus for being engaged at least to the nose of the face, comprising:
a) a flexible base having front and rear sides defining a thickness, upper and lower edges defining a length, and right and left edges defining a width;
b) adhesive engaged to the flexible base to stick the apparatus to the nose; and
c) a vapor medication engaged to the flexible base to be delivered to at least one or more of the nasal passages, sinus, throat and lungs.

2. The apparatus of claim 1, wherein the flexible base when attached to the nose extends beyond the tip of the nose and beyond the nostrils of the nose and forms at least a portion of a chimney about the nostrils and tip of the nose.

3. The apparatus of claim 1, wherein the flexible base when attached to the nose extends beyond the tip of the nose and beyond the nostrils of the nose and forms at least a portion of a tube about the nostrils and tip of the nose.

4. The apparatus of claim 2, wherein the rear side of the flexible base faces toward the nose and face, wherein the front side of the flexible base faces away from the nose and face, and wherein medication passes from the rear side of the flexible base, into the chimney portion, up the chimney portion, and into the nasal passages.

5. The apparatus of claim 3, wherein the rear side of the flexible base faces toward the nose and face, wherein the front side of the flexible base faces away from the nose and face, and wherein medication passes from the rear side of the flexible base, into the tube portion, up the tube portion, and into the nasal passages.
